(19) United States
(12) Patent Application Publication
(10) Pub. No.: US 2020/0039812 A1
(43) Pub. Date: Feb. 6, 2020

(54) A MICROFLUIDIC ANALYTICAL PLATFORM FOR AUTONOMOUS IMMUNOASSAYS

(71) Applicant: Xinyu LIU, Verdun (CA)

(72) Inventors: Xinyu LIU, Verdun (CA); Hao FU, Montreal (CA)

(21) Appl. No.: 16/339,769

(22) PCT Filed: Oct. 6, 2017
(86) PCT No.: PCT/CA2017/051198

§ 371 Date: Apr. 5, 2019

Related U.S. Application Data
(60) Provisional application No. 62/405,492, filed on Oct. 7, 2016.

(51) Int. Cl. B81B 3/00; G01N 33/543

(52) U.S. Cl. CPC B81B 3/0024; G01N 33/543; B81B 2201/058; B81B 2201/032; B81B 2201/0214

(57) ABSTRACT

It is provided a microfluidic analytical device and platform for autonomous immunoassays such as ELISA comprising a porous layer having at least one slot therein and a porous arm extending from the porous layer and pivotable about the arm's root, the porous arm being pivotable between an off position wherein the porous arm is spaced away from the slot and an on position wherein the porous arm is disposed in the slot and the hydrophilic element spans the slot to define a fluid flow path across the slot; a heat-responsive shaped memory polymer (SMP) disposed underneath the porous layer, the SMP being elastically deformable in response to being heated to move the porous arm between the on and off positions; and a heat source in heat-conducting contact with the SMP to elastically deform the SMP.

### CROSS-REFERENCE TO RELATED APPLICATIONS

The present application claims benefit of U.S. Provisional Application No. 62/405,492 filed Oct. 7, 2016, the content of which is hereby incorporated by reference in its entirety.

### TECHNICAL FIELD

It is provided a microfluidic analytical device and platform for autonomous immunoassays such as ELISA.

### BACKGROUND

Point-of-care (POC) biosensors are designed for rapid and sensitive detection of molecular markers in sample fluids, and could improve personal healthcare, ensure food safety, and monitor environmental safety. Microfluidic paper-based analytical devices (μPADs) increasingly become one of the most significant candidates among POC diagnostic approaches and provide an inexpensive, easy-to-use, and safe biosensing platform [1]. Enzyme-linked immunosorbent assay (ELISA), a widely-used assay for clinical diagnosis, has been achieved on μPADs to make health-related applications accessible [2, 3]. However, these ELISA μPADs requires human interventions, such as repeated pipetting of reagents, capturing the assay readout signal using a scanner or camera, and analyzing the imaged results through software, and thus certain level of operator skills, which restrains these paper-based devices from being used by untrained or less-skilled users. It is highly desired to realize full automation of ELISA on a μPAD, which will eliminate human interventions and assure the developed μPAD to provide completely ASSURED (proposed by the World Health Organization (WHO); affordable, sensitive, specific, user-friendly, rapid and robust, equipment-free, and deliverable to the end user) tests [1].

The inherent capillarity of the porous paper exempts μPADs from pumping instrument for fluid manipulation, but controllable fluid valves on paper substrates are still needed to realize assay automation. Over the past several years, paper-based microfluidic valves controlled by mechanical means have been utilized for programmable control of fluid flows on μPADs [4-6]. Although these methods eliminated the repeated pipetting of reagent solutions, manual operations are still required for valve actuation. Most recently, it was reported a new type of normally-open and normally-closed magnetic timing valves for fluid control on paper-based microfluidics [7]. This design functionalizes a mechanical cantilever valve with magnetic nanoparticles, making it controllable by magnetic forces. This saves the manual operations for turning on or off the valve. It was demonstrated automatic single-step fluidic operations commonly used in multistep assays. However, each magnetic valve requires an off-chip, relatively bulky electromagnet for actuation, and the integration of multiple magnetic valves will lead to a large foot print (>10 cm×10 cm for a four-valve device) of the μPAD. Yager and co-workers also demonstrated localized valves on μPADs with compressed sponges as actuators in an integrated toolkit [8]. An untrained user can perform ‘sample-in-answer-out’ (SIAO) ELISA using a μPAD integrating such valves. However, the design includes many moving parts (e.g., test strips, sponge-based valves, and glass fiber actuation channels) which could limit the reliability of the device manufacturing and operation. Furthermore, the toolkit only provides qualitative diagnostic answers directly, and subsequent off-chip analysis of the colorimetric result is needed for quantitative readout. For all the controllable fluidic valves that have been developed, they can only unidirectionally switch from the ‘on’ to ‘off’ or ‘off’ to ‘on’ states, and no design can subsequently realize turn-on (turn-off) and turn-off (turn-on) operations by the same valve.

There is thus still a need to be provided with an improved system and device for autonomous ELISA.

### SUMMARY

One aim of the present disclosure is to provide a microfluidic analytical device, comprising a porous layer having at least one slot therein and a porous arm extending from the porous layer and pivotable about the arm's root, a distal end of the porous arm having a hydrophilic element, the porous arm being pivotable between an off position wherein the porous arm is spaced away from the slot and an on position wherein the porous arm is disposed in the slot and the hydrophilic element spans the slot to define a fluid flow path across the slot; a heat-responsive shaped memory polymer (SMP) disposed underneath the porous layer and abutting against the porous arm, the SMP being elastically deformable in response to being heated to move the porous arm between the on and off positions; and a heat source in heat-conducting contact with the SMP to elastically deform the SMP.

In an embodiment, the porous layer is selected from the group consisting of porous cellulose paper, porous hydrophilic fabrics, porous nitrocellulose paper and membrane, porous glass microfiber membrane, and porous carbon nanofiber membrane

In another embodiment, the porous layer comprises fluid-impermeable barriers that define boundaries of hydrophilic regions; said hydrophilic regions comprises a fluid channel, a reagent storage zone, and a test zone; said fluid channel connects said reagent storage zone and said test zone.

In a further embodiment, the test zone comprises an immobilized analyte binder.

In an additional embodiment, the slot disconnects said fluid channel.

It is also provided an analytical system, comprising a printed circuit board having heating resistors disposed thereon, the printed circuit board being operable to energize the heating resistors to generate heat therefrom; and a microfluidic analytical device, comprising a porous layer disposed over the printed circuit board, the porous layer having a slot therein and a porous arm extending from the porous layer and pivotable about the arm's root, a distal end of the porous arm having a hydrophilic element, the porous arm being pivotable between an off position wherein the porous arm is spaced away from the slot and an on position wherein the porous arm is disposed in the slot and the hydrophilic element spans the slot to define a fluid flow path across the slot; and a heat-responsive shaped memory polymer (SMP) disposed beneath the porous layer and above the printed circuit board, the SMP abutting against the porous arm and being in heat-conducting contact with the heating resistors, the SMP being elastically deformable in response to being heated by the heating resistors to move the porous arm between the on and off positions.

In an embodiment, the analytical system described herein further comprises a light-emitting diode (LED) and a red-green-blue color sensor for measuring the output signal of the assay.

In another embodiment, the analytical system described herein further comprises a liquid crystal display (LCD) screen for displaying the signal of the assay.

In a further embodiment, the analytical system described herein further comprises a wireless communication module for transmitting assay result data to a cell phone or a computer.

In an embodiment, the wireless communication module is a Bluetooth communication module.

It is also provided a method of analyzing a fluid analyte, comprising heating a porous arm to fold the porous arm into a slot, a hydrophilic portion of the porous arm spanning the slot and forming a fluid flow path across the slot; and conveying a fluid reagent over the hydrophilic portion of the folded porous arm across the slot and into a test zone; and analyzing the fluid analyte in the test zone.

In an embodiment, the fluid analyte is selected from the group consisting of antigen and antibody markers.

In a further embodiment, the method described herein is for a direct or sandwich ELISA.

### BRIEF DESCRIPTION OF THE DRAWINGS

Reference will now be made to the accompanying drawings.

FIG. 1 illustrates a schematic representation of (a) A μPAD with SMP-actuated micro-valves for autonomous ELISA; and in (b) a protocol of direct ELISA.

FIG. 2 represents operation of a SMP-actuated valve showing in (a) the valve initial state is OFF; and in (b)-(d) after activations #1 and #2, the reagent is transferred from the storage zone to the test zone.

FIG. 3 is showing in (a) a photograph of an exemplary portable platform that can accommodate the μPAD, activate the valves, and readout the colorimetric signal; and in (b) an explored view of the platform architecture of an exemplary embodiment with the major components.

FIG. 4 is a schematic representation of a functionalization of the test zone in the wax-printed μPAD for covalent binding proteins with amino groups.

FIG. 5 represents the characterization of functionalized test zone using FTIR.

FIG. 6 represents the normalized mean grayscale intensity values for the detection of rabbit IgG on μPAD with functionalized test zone and unmodified μPAD as direct ELISA.

FIG. 7 represents the light transmittance signals measured from the test zone at each ELISA step by the RGB color sensor.

FIG. 8 relates to information on direct ELISA for rabbit IgG in PBS in which in (a) are photographs of test zones of direct ELISA for rabbit IgG at different IgG concentrations; and in (b) are calibration curves of the mean grayscale intensity signal versus the IgG concentration on the test zone.

FIG. 9 represents the evaluation of different quantification methods with RGB color sensor, scanner, and camera based on the direct ELISA for rabbit IgG.

FIG. 10 shows displays calibration plots of modeling experiment for the mean grayscale intensity from RGB color sensor to the mean grayscale intensity from scanner.

FIG. 11 shows an exemplary interface of a control pad on a smartphone for the Bluetooth module.

FIG. 12 provides information related to a sandwich ELISA for rat TNF-α in PBS and rat tissue extraction showing in (a) a calibration curve of the mean grayscale intensity signal versus the TNF-α concentration on the test zone; and in (b) the detection performance for TNF-α in extraction from rat vocal tissue, in standard TNF-α sample and device described herein.

### DETAILED DESCRIPTION

In accordance with the present invention, there is provided a fully-automated, paper-based microfluidic platform for autonomous ELISA. The porous layer of the microfluidic analytical device is cellulose paper. In other possible embodiments, the porous layer can be selected from the group of porous hydrophilic fabrics, porous nitrocellulose paper and membrane, porous glass microfiber membrane, and porous carbon nanofiber membrane.

A heat-responsive shape memory polymer (SMP) is integrated, for the first time, onto a μPAD for actuating a paper cantilever beam to serve as a bidirectional valve. The SMP-based valves are triggered by individual heating resistors fabricated on a printed circuit board (PCB) underneath the paper device, are thus small-sized and allow integration of several valves on a μPAD with small footprint. Based on this design, an automatically-operated μPAD was fabricated, integrating multiple SMP-based valves, for performing autonomous direct and sandwich ELISAs. The platform is integrated with several functional components: (i) a microcontroller for controlling the valves and performing automated assay operations; (ii) a flexible PCB with heating resistors for programmed triggering of the valves on the μPAD; (iii) a custom-made colorimetric reader, including a light-emitting diode—LED (as the light source) and a red-green-blue (RGB) color sensor (as the colorimetric reading unit), for quantitative readout of the final colorimetric signal from the μPAD; (iv) a liquid crystal display (LCD) screen for displaying the quantitative result; and (v) a Bluetooth module for wireless data transmission of the testing result. A self-checking mechanism for valve malfunction by detecting the light transmittance difference can also be incorporated in the device to detect the failure of the μPAD operation and remind a user to replace the failed μPAD with a new one. This user-friendly device requires no human intervention during the multi-step ELISAs. Besides the standard calibration experiments, it is demonstrated the effectiveness of the platform using real rat samples for detection of TNF-α, and achieved testing results comparable to those of standard ELISAs.

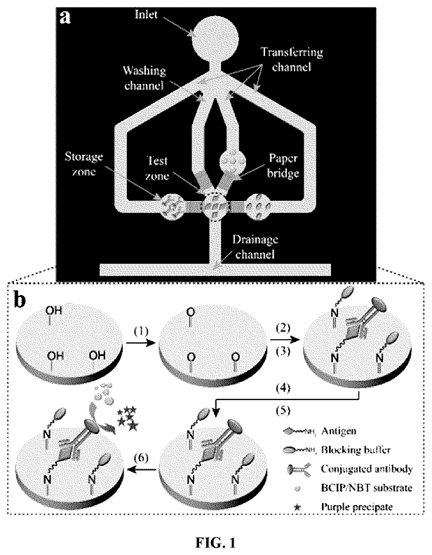

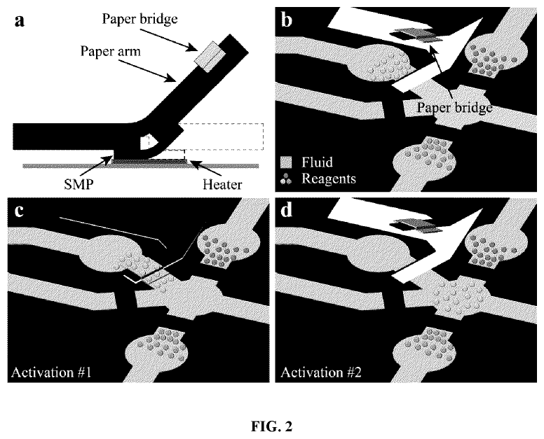

FIG. 1a shows the μPAD with SMP-actuated values for direct ELISA. All reagents were stored in the storage zones and transferred to the test zone by buffer flows from an inlet. The SMP-actuated valves could connect and disconnect the storage zones and the test zone, and thus controlled the sequence and timing of reagent transfers based on the autonomous ELISA protocol. A heat-responsible SMP deforms from its permanent shape to a temporary shape at a temperature above the switching transition temperature (Tₜᵣₐₙₛ), and maintains its temporary shape by cooling down to a temperature below Tₜᵣₐₙₛ. When heated up again above Tₜᵣₐₙₛ, the SMP will be transformed back to its permanent shape. Due to this heat-responsible response, self-assembly robots with SMPs have been reported to accomplish a localized and sequential three-dimensional folding process [9, 10]. With a piece of heat-responsible SMP attached to a foldable paper cantilever arm (FIG. 2a), a thermally-controlled fluidic valves can be fabricated, and this design eventually led to a μPAD capable of autonomous multi-step ELISA.

Each valve (FIG. 2a) included a paper arm pre-cut from the μPAD, a sheet of SMP (attached to the foldable root of the paper arm) which deforms via joule heating by a heating resistor underneath it, and a hydrophilic tissue-paper bridge attached to the paper arm tip. The SMP sheet was attached to the paper arm root with a pre-cut hinge, and the paper arm was initially bent up at 45° (FIG. 2b). A polyolefin (PO, Tₜᵣₐₙₛ=95° C.) was utilized as the SMP material. Following the optimized shape of a copper heating resistor [9], the width and length of the PO sheet were experimentally determined to be 6 mm and 12 mm, respectively. After the comparison of different PO sheets for the best valving performance, the SMP model of RNF-100 1″×4′ BLK (thickness=0.89 mm) was chose for μPAD fabrication, which provides an appropriate response time and the highest success rate (Table 1).

TABLE 1
Comparison of three kinds of SMP-actuated valves (n = 15)
for valving performance on the device.

| | Time of | Time of | Success |
|---|---|---|---|
| SMP-actuated | activation #1 | activation #2 | rate |
| valves | (s) | (s) | (%) |
| 3/8 IN × 4 FT BLK | 13.3 ± 1.7 | 27.8 ± 2.7 | 33 |
| (thickness = 0.64 mm) | | | |
| RNF-100 1″ × 4′ BLK | 22.7 ± 3.7 | 45.9 ± 10.4 | 93 |
| (thickness = 0.89 mm) | | | |
| RNF-100 2″ × 4′ BLK | 30.1 ± 5.4 | 103 ± 23.9 | 73 |
| (thickness = 1.14 mm) | | | |

Time of activation #1 was the heating time of copper traces from starting to turning off. Time of activation #2 was the heating time of copper traces from starting to turning off after transferring the liquid for 1 minute. The successful accomplishment of both activation #1 and activation #2 was counted positive for the success rate collection

The SMP flattened the paper arm once heated for 25 s (activation #1, ON) for turning on the valve, and connected the channel. Then, the heater was turned off to maintain the SMP flat (i.e., maintain the channel connection), and a buffer fluid in the upstream of the valve transferred the stored reagent to the test zone (FIG. 2c). Finally, the SMP was heated again for 55 s (activation #2, OFF) to bring it back to its initial shape and turn off the valve (FIG. 2d). One can see that a single valve can perform ‘turn-on’ and ‘turn-off’ operations sequentially.

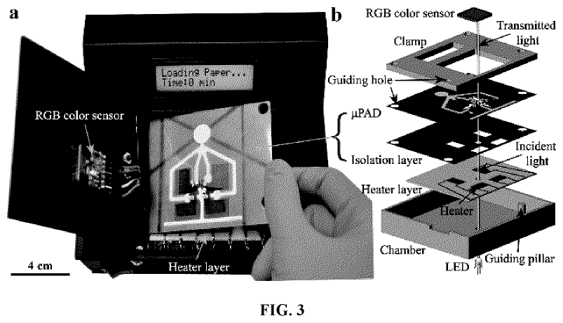

A μPAD (FIG. 3) consists of one layer of lamination plastic (for reducing evaporation) and a paper sheet of patterned microfluidic channels with paper valves attached with SMP sheets. One piece of isolation paper printed full of wax with laser-cut openings is placed under the μPAD to prevent liquid leakage from the channels down to the heating resistor layer. During each assay, a clamp was placed on top of the μPAD to secure intimate contact between the SMP sheets and the copper heating resistors (FIG. 3).

To automatically operate the μPAD, an integrated electronic holder (FIG. 3) was developed for programmed valve activation and colorimetric signal readout from the μPAD. The holder includes a microcontroller circuit, a patterned heating resistor layer, a plastic casing, a LED light source, a RGB color sensor, a LCD screen, and a Bluetooth communication module. The copper heating resistor traces are 0.5 mm wide and were patterned by wet etching. Each resistor trace is in a serpentine pattern to maximize heating efficiency, then was bonded to a piece of hardboard and fixed onto the chamber in our device. Each trace is switched by a transistor controlled by the microcontroller, and supplied with 1.2 W power in a parallel step-down circuit as heat source. A white LED (λₘₐₓ=550 nm) and an IC-based RGB color sensor are used as the light source and the photodetector, respectively. The color sensor contains an array of 3×3 red-filtered, green-filtered, and blue-filtered photodiodes, and provides a digital readout of RGB sensing values. The LED and the color sensor are installed coaxially with the test zone of the μPAD for transmission-based colorimetric measurement.

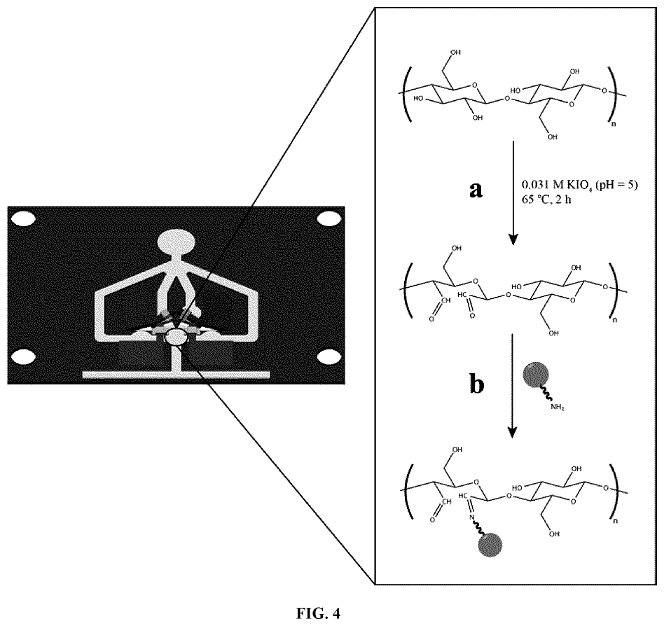

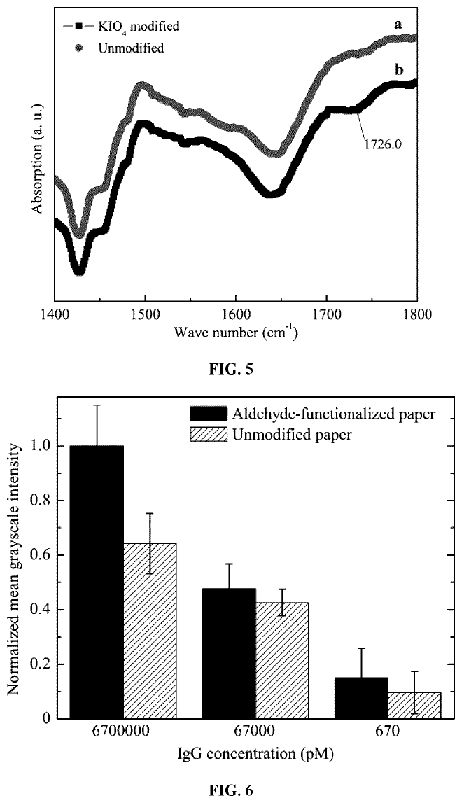

A Xerox 8570DN inkjet printer was used for photo-quality printing of wax-based solid ink on Whatman No. 1 chromatography paper to form patterns of microfluidic channels. Then, the paper was placed on a hot plate for 30 s at 120° C. for melting the wax to form hydrophilic channels of the μPAD. The test zone (6 mm in diameter) of μPAD was oxidized for aldehyde-functionalization by spotting 3 μL of 0.031 M KIO₄ (pH=5) solution every 5 minutes for 2 hours and baking at 65 celcius (FIG. 4). After the functionalization, 10 μL of deionized water (DI H₂O) was added twice to the test zone for washing away the residual oxide. Finally, the paper was dried in a desiccator for at least 12 hours before use. The aldehyde groups in the test zone can effectively immobilize proteins with their amino groups to the cellulose skeleton of the paper through the Schiff-base linkage. The oxidization process was monitored by Fourier transform infrared spectroscopy (FTIR). From the infrared spectra, the characteristic absorption band of aldehyde group in the oxidized cellulose zone appeared at 1726 cm⁻¹ (FIG. 5) due to the stretching vibration of C═O double bond. We also verified the performance of aldehyde-functionalized paper using direct ELISA (FIG. 6).

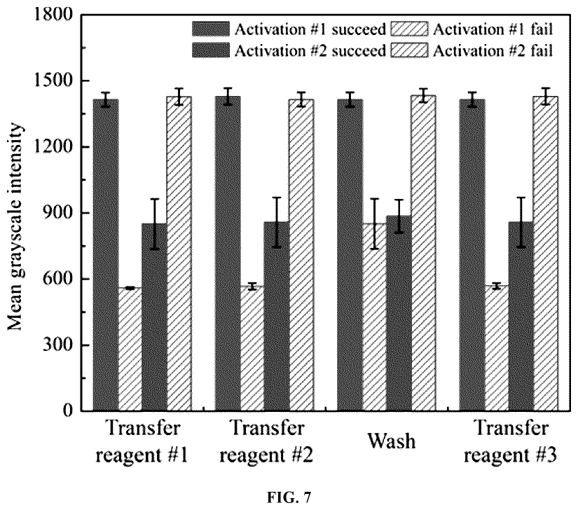

Through experiments, the operation success rate of the SMP-actuated valves was found to be 93% (n=60). To monitoring the valve operations and eliminate the failed ones, a self-checking mechanism was also established in the device for valve malfunction. The malfunction of a valve fails to connect the channel and transfer the liquid from the inlet. Thus through detecting the light transmittance difference of the test zone in dry and (semi-)wet states, the RGB color sensor monitors the mean grayscale intensity in the test zone right after the valve is ON (activation #1) and one minute after the valve is OFF (activation #2). Due to the prominent scattering capacity of liquid, the light transmittance of the test zone will be significantly enhanced if the valve successfully connects the channel and transfers the reagent-carrying fluid to the test zone. This can be confirmed by the transmission readout of the test zone by the RGB color sensor (FIG. 7). For all the ELISA steps except the washing step, the self-checking mechanism identifies the valve malfunction by detecting light transmittance difference of the test zone in dry and (semi-)wet states (as each step is performed after 10-minute incubation, which dries the test zone). For the washing step to remove the unbounded antibodies by PBS, it is conducted only after one-minute incubation, which makes the test zone only slightly less wet (what we call the ‘semi-dry’ state). The RGB color sensor is still capable of distinguishing light transmission difference of the test zone between the wet and semi-wet states for one minute (FIG. 7).

### Example I

### Materials and Reagents

Whatman No. 1 chromatography paper, bovine serum albumin (BSA), rabbit IgG, anti-rabbit IgG (alkaline phosphatase conjugated), anti-rabbit IgG (fluorescein isothiocyanate conjugated), 3,3′,5,5′-tetramethylbenzidine (TMB) (99%), BCIP®/NBT, Tween® 20, 10× phosphate buffered saline (PBS), and potassium periodate were purchased from Sigma-Aldrich and used without further purification. Recombinant rat TNF-α, rat anti-TNF-α antibody, and horseradish peroxidase (HRP)-conjugated streptavidin were purchased from Abcam (Toronto, ON). Biotinylated anti-Rat TNF-α was purchased from BioLegend (San Diego, Calif.). White LED and polyolefin (PO) were purchased from Digi-Key Cooperation (Thief River Falls, Minn., USA). Arduino UNO as microcontroller and 16×2 LCD as display were purchased from RobotShop Inc. (Mirabel, QC, Canada). Pyralux® (LF7062) copper-coated polymide film was got as sample from DuPont. Ferric chloride was purchased for etching the copper from MG Chemicals. RGB color sensor (TCS34725) was purchased from Adafruit (New York, N.Y., USA). Scotch plastic thermal laminating pouches were purchased from 3M.

### Example II

### Visualization and Imaging for the Test Zone on Paper

Uniform light was the key for reproducibility when imaging taken at different times of the day. For cellphone camera, different light condition (high noise) lead to different colorimetric intensity values for that same picture. A mini photostudio was used to filter ambient light to be uniform at the time of image capture. This method achieved higher reproducibility on colorimetric intensity values for cellphone camera.

### Example III

### Consistency of Detection Method

For paper-based ELISA, the reaction substrate for the assay is cellulose paper. Therefore, proteins (antigens, antibodies, etc.) are adsorbed to the cellulose fiber throughout the whole paper thickness (180 μm for Whatman No. 1 chromatography paper). Current detection methods for paper-based ELISA mostly utilize scanner or cellphone to capture images and then analyze the mean grayscale intensity using ImageJ or other software. Both of the methods only reflect the color intensity of paper surface, and neither detection method for paper-based ELISA will not read the complete coloration values throughout the whole paper thickness. However, based on the detection mechanism in our device, RGB color sensor can reflect the color intensity throughout the whole paper thickness.

### Example IV

### Approximation for Measuring the Transmission of Light Through Paper

In the device described herein, the detection mechanism imitated the ultraviolet-visible spectroscopy restricting the light path from the LED to detector (RGB color sensor in our device) through the test zone on paper-based microfluidics in a fixed orientation. One optical mode [19] which adequately took consideration of refraction by paper fibers, attenuation of non-assay due to absorbance of the sample, and the boundary transmission factors for the air-paper and paper-air interfaces, has been set up in equation (1). In this mode, the total transmittance (T) varies corresponding to the different factors. I (W/cm²) is the intensity transmitted from the LED to the RGB color sensor through the test zone with a colorimetric assay, I₀ (W/cm²) is the source intensity, αₛₐₘₚ (cm⁻¹) is the factor for attenuation (both scattering and absorption) caused by the sample in the test zone, and z is the thickness of the paper. Moreover, ε (M⁻¹ cm⁻¹) and c (M) are the molar extinction coefficient and concentration of analyte. In this equation, c is the apparent concentration of sample causing the colorimetric results, so it also can be defined as I_{valid} (the valid colorimetric intensity shown in the paper; also has a non-linear regression using the Hill Equation with the real concentration of sample).

$$T = \frac{I}{{I}_{0}} = {T}_{C} {10}^{- {α}_{samp} z} {10}^{- ɛcz} \qquad ( 1 )$$

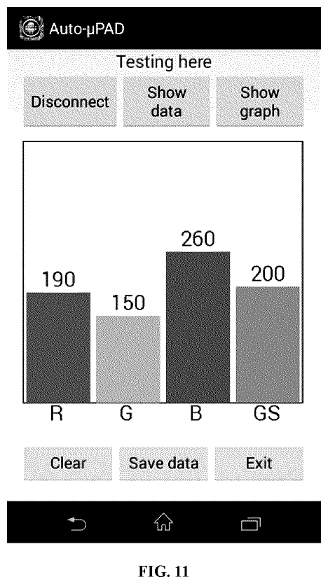

For simplifying the calculation method, total transmittance detected by the RGB color sensor was confirmed has a linear relationship with I_{valid} in the test zone in a small intensity interval overlapping the colorimetric changing in P-ELISA. According to some previous works [20, 21], the analytes were detected directly using scanner by quantifying changes in color intensity (I_{valid}) caused by the coloration of the analyte on the surface. A modeling experiment was carried out using purple dye in 2-fold dilutions (1:1 to 1:32) which has the similar color with colorimetric results of ELISA to test the signal response detected by RGB color sensor and scanner; PBS was used as a negative control. FIG. 10 proved that during the same intensity interval with colorimetric results for ELISA, the signal detected by RGB color sensor and scanner has an excellent linear dependence.

### Example V

### Bluetooth Module

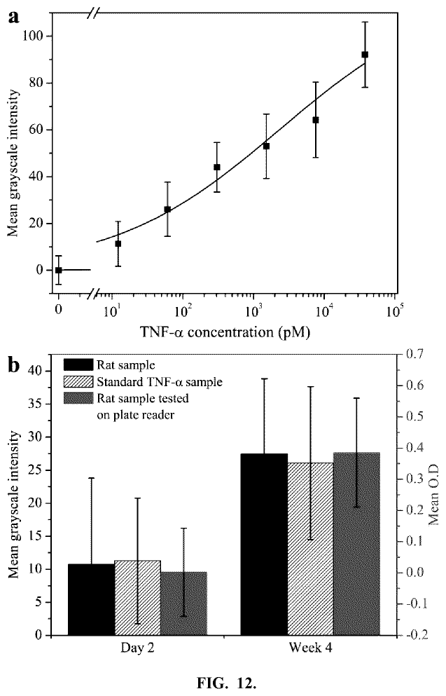

FIG. 11 illustrates the wireless transmission architecture in the platform described herein, from the device, to a PC or a smart phone, and finally to a remote cite. The PC software and smart-phone APP both can open the Bluetooth port of the device and trigger the automated assays, and receive data via wireless transmission. The data not only can be transmitted from the potentiostat to a PC through the USB, but further to a remote site (e.g., centralized laboratory or public health database) via the Internet (by a PC or a smartphone) or the mobile network (by a smartphone), for tele-diagnosis or healthcare data collection.

### Example VI

### Autonomous Direct ELISA

Using the platform, the autonomous direct ELISA was demonstrated for the detection of rabbit IgG on our device. The entire system operation can be visualized using food dyes mimicking the stored reagents. Before the assay, the test zone in the μPAD was functionalized using KIO₄ for amplifying the colorimetric signal. After that, following the protocol of direct ELISA [2] optimized for our lateral-flow μPAD, rabbit IgG antigen (3 μL at different known concentrations) was immobilized to the test zone and used 1×PBS as a negative control. Blocking buffer (3 μL of 0.5% (v/v) Tween-20 and 10% (w/v) BSA in PBS), alkaline phosphatase (ALP)-conjugated antibody (3 μL of 1:10 dilution of the antibody solution in PBS), and 5-bromo-4-chloro-3-indolyl phosphate and nitro blue tetrazolium substrate (3 μL of 4.59 mM BCIP, 3.67 mM NBT, 50 mM MgCl₂ in 1 M Tris buffer, pH 9.5) were pipetted onto their respective storage zones (6 mm in diameter) of the μPAD. Finally, the μPAD was assembled manually.

To run an ELISA, a user mounts the μPAD onto the chamber in the device, and adds 250 μL of PBS on the buffer inlet of the μPAD, closes the platform door (for minimizing evaporation and maintaining a dark chamber for colorimetric measurement), and presses a button on the platform to start the assay. Automated operation of the four paper system is controlled by the microcontroller following a pre-programmed protocol: (i) after 3 minutes standing for wetting out upstream paper channels, the blocking buffer is transferred from the storage zone to the test zone, and the test zone is then incubated for 10 minutes; (ii) the ALP-conjugated antibodies are transferred to the test zone for labeling the immobilized antigens, and incubated for 1 minute; (iii) the test zone is washed by PBS for removing the un-bound antibodies, and incubated for 10 minutes; (iv) the BCIP/NBT substrate is transferred from to the test zone, and incubated for 30 minutes for signal amplification; and (v) the microcontroller lights up the LED for shining incident light to the test zone, and the RGB color sensor measures the transmitted colorimetric signal and transmits the 16-bit digital data to the microcontroller for result display on the LCD. During the assay, the self-checking mechanism for valve malfunction continuously monitors the operations of all the valves and reports error if a failure occurs.

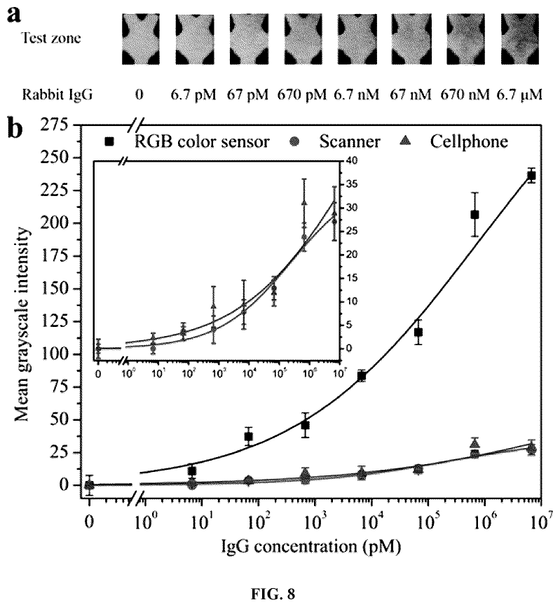

A direct ELISA was carried out for the detection of rabbit IgG in 10-fold dilutions (6.7 mM to 6.7 pM), and the calibration results of the measured colorimetric intensity vs. the IgG concentration are shown in FIG. 8. For comparing the performance of the RGB color sensor with that of other colorimetric readout methods, the same assay results was captured by a desktop scanner (CanoScan LiDE 210 scanner, CANON Inc.; set to color photo scanning, 300 dpi resolution) and a cellphone (Xperia™ Z cellphone, SONY Electronics Inc.; 4128×3096 pixels). The images were analyzed in mean grayscale intensity using ImageJ. All the data was fitted into sigmoidal curves using the Hill Equation (FIG. 8b), and the limits of detection (LOD) were calculated.

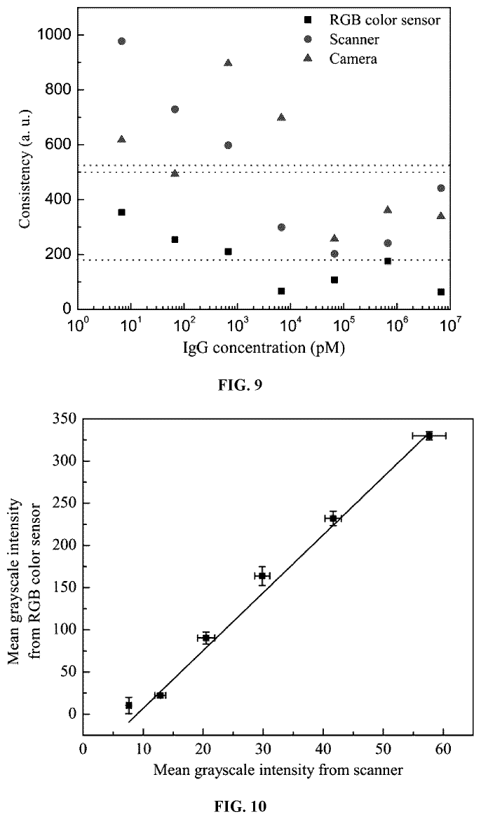

For coefficient of determination fitted with the Hill Equation, the best-fit to the worst-fit method is: RGB color sensor (0.993), scanner (0.970), and cellphone (0.894). The continuous flashing of scanner for capturing images leads to the optical correction for true colorimetric intensity of assay detection on the μPAD. Thus it has a slight distortion for the true coefficient of determination. For cellphone, although we set up a shelter to filter the ambient light into uniform light source for taking the cellphone images (Supporting Information), the images were still suffers from ambient reflections, camera distortion, and low contrast. Therefore, compared to the desktop scanner and cellphone, the RGB color sensor supplies the least variance for quantifying the colorimetric signal. For the LOD, the readouts bearing the lowest LOD to the highest LOD are the ones from: RGB color sensor (27 pM), scanner (255 pM), and cellphone (836 pM). Also, the RGB color sensor provides the highest sensitivity for the assay. Besides, the detection error would influence the results least compared with the scanner and cellphone (FIG. 9). Although the intense flash of scanner improves the photocopy appearance, it reduces the intensity difference of light and dark colors and thus the sensitivity of the assay because of the strong background light. Cellphone-based camera enhances the convenience for users taking photographs, but some functionalities of the cellphone (e.g., ISO self-adjustment, and color rendition or distortion) is not appropriate for measuring colorimetric results in our assay. In our device, the detection mechanism is based on colorimetric results in the test zone with incident light and transmitted light provided by the LED. The optical mode was simplified [11] and approximated it into our detection mechanism with better reflection of the autonomous ELISA (FIG. 10).

### Example VII

### Autonomous Sandwich ELISA for Real Rat Sample

Direct ELISA is a rapid (with fewer steps) and straightforward assay for testing the performance of our platform. Sandwich ELISA on the proposed platform was also demonstrated since it is more widely used for testing real complex clinical samples and has higher sensitivity and specificity. Based on the same architecture of the μPAD, its design was modified by increasing the number of reagent storage zones so that a sandwich ELISA with more reaction steps (thus more types of reagents) can be performed using the paper device described herein. Sandwich ELISA for rat TNF-α in 5-fold dilutions (59 nM to 19 pM) was performed first on the device generating the calibration curve. Before the assay, anti-rat TNF-α (3 μL of 1:10 dilution of the antibody solution in PBS) as the capture antibody was immobilized in the oxidized test zone, and then rat TNF-α (3 μL) was spotted to the test zone for binding with the capture antibody. PBS was used as a negative control. Pre-mixed biotin-conjugated anti-rat TNF-α (1.5 μL of 1:5 dilution of the antibody solution in PBS) as secondary antibody, HRP streptavidin (1.5 μL of 1:5 dilution of the enzyme in PBS), HRP substrate (3 μL of 4 mM TMB in DMSO and 0.05 M phosphate-citrate buffer with trace amount of fresh 30% hydrogen peroxide, pH 5.0), and stop solution (3 μL of 4 mM sulfuric acid) were pre-stored in the storage zones. Then, the steps were operated by the same customized program as the above-mentioned direct ELISA. Finally, the test zone was quantified by the RGB color sensor and the results were fit into the Hill Equation (FIG. 12a). For confirming the feasibility of the pre-mixed solution of secondary antibody and HRP streptavidin in the sandwich ELISA, the assay was also carried out by adding the secondary antibody and HRP streptavidin respectively. By comparing the different approaches, two kinds of assay results revealed no difference via student's t-test (p=0.132; n=7).

For investigating the potential application of the device for clinical test, the sandwich ELISA was demonstrated for detection of TNF-α in fluid extraction from rat vocal fold tissue over 4 weeks after vocal fold surgery, and compared the detection performance with traditional ELISA kit on plate reader. First, a standard TNF-α rat ELISA kit experiment quantified by a plate reader was conducted to measure the concentrations of TNF-α in the tissue extractions to be 21 pM and 77 pM for day 2 and week 4 after vocal fold surgery, respectively (FIG. 12b). Then, the same batch of extraction samples and the standard rat TNF-α (21 pM and 77 pM) were tested by the platform (FIG. 12b). The data from the standard ELISA and the enclosed platform were analyzed via Student's t-test (Table 2).

TABLE 2
Comparison of the detection performance for TNF-α in extraction from
rat vocal fold tissue after vocal fold surgery and in standard recombinant
rat TNF-α on the device (n = 5) via the Student's t-test

| | p value | Indication |
|---|---|---|
| Rat sample after surgery over 2 days | 0.96 | No obvious |
| vs. | | difference |
| standard TNF-α sample (21 pM) | | |
| Rat sample after surgery over 4 weeks | 0.85 | No obvious |
| vs. | | difference |
| standard TNF-α sample (77 pM) | | |
| Rat sample after surgery over 2 days | 0.06 | Relative |
| vs. | | obvious |
| rat sample after surgery over 4 weeks | | difference |

There was no obvious difference for between the detection of TNF-α for rat sample (after surgery over 2 days and 4 weeks) and standard sample. Also the device could distinguish the rat sample after surgery over 2 days and 4 weeks. As shown in FIG. 12b, compared with the results from the standard ELISA on the same extraction samples, the proposed device (black columns in FIG. 12b) provides comparable testing results (p>0.05). This comparison result demonstrates the feasibility of the device on real rat sample test.

For the first time, a self-contained and self-regulated paper-based platform was developed for autonomous ELISA. This user-friendly device requires no human intervention (e.g., repeated pipetting of reagents, capturing and measuring the calorimetric results) during the multi-step assays, and enables sample-in-answer-out operations. Direct ELISA of rabbit IgG was performed to evaluate the device performance, and indirect ELISA of TNF-α in animal samples was also carried out as a practical application. Besides ELISA tests, the proposed platform can also be readily adapted for other single- and multi-step assays, such as detection of glucose [12], other proteins (e.g., bovine serum albumin) [13], uric acid [14], lactate [15], pH [16], pathogenic bacteria (e.g., Pseudomonas aeruginosa, Staphylococcus aureus, E. coli O157:H7, Salmonella typhimurium, and L. monocytogenes) [17, 18].

### REFERENCES

1. Martinez, A. W., et al., Diagnostics for the developing world: microfluidic paper-based analytical devices. Analytical chemistry, 2009. 82(1): p. 3-10.
2. Cheng, C. M., et al., Paper-Based ELISA. Angewandte Chemie International Edition, 2010. 49(28): p. 4771-4774.
3. Liu, X., et al. A portable microfluidic paper-based device for ELISA. in Micro Electro Mechanical Systems (MEMS), 2011 IEEE 24th International Conference on. 2011. IEEE.
4. Li, X., et al., Paper-Based Micro fluidic Devices by Plasma Treatment. Analytical chemistry, 2008. 80(23): p. 9131-9134.
5. Martinez, A. W., et al., Programmable diagnostic devices made from paper and tape. Lab on a Chip, 2010. 10(19): p. 2499-2504.
6. Glavan, A. C., et al., Rapid fabrication of pressure-driven open-channel microfluidic devices in omniphobic RF paper. Lab on a chip, 2013. 13(15): p. 2922-2930.
7. Li, X., P. Zwanenburg, and X. Liu, Magnetic timing valves for fluid control in paper-based microfluidics. Lab on a Chip, 2013. 13(13): p. 2609-2614.
8. Toley, B. J., et al., A versatile valving toolkit for automating fluidic operations in paper microfluidic devices. Lab on a Chip, 2015.
9. Felton, S. M., et al., Self-folding with shape memory composites. Soft Matter, 2013. 9(32): p. 7688-7694.
10. Felton, S. M., et al. Robot self-assembly by folding: A printed inchworm robot. in Robotics and Automation (ICRA), 2013 IEEE International Conference on. 2013. IEEE.
11. Ellerbee, A. K., et al., Quantifying colorimetric assays in paper-based microfluidic devices by measuring the transmission of light through paper. Analytical chemistry, 2009. 81(20): p. 8447-8452.
12. Martinez, A. W., et al., Patterned paper as a platform for inexpensive, low-volume, portable bioassays. Angewandte Chemie International Edition, 2007. 46(8): p. 1318-1320.
13. Martinez, A. W., S. T. Phillips, and G. M. Whitesides, Three-dimensional microfluidic devices fabricated in layered paper and tape. Proceedings of the National Academy of Sciences, 2008. 105(50): p. 19606-19611.
14. Li, X., J. Tian, and W. Shen, Progress in patterned paper sizing for fabrication of paper-based microfluidic sensors. Cellulose, 2010. 17(3): p. 649-659.
15. Dungchai, W., O. Chailapakul, and C. S. Henry, Use of multiple colorimetric indicators for paper-based microfluidic devices. Analytica Chimica Acta, 2010. 674(2): p. 227-233.
16. Abe, K., K. Suzuki, and D. Citterio, Inkjet-printed microfluidic multianalyte chemical sensing paper. Analytical chemistry, 2008. 80(18): p. 6928-6934.
17. Li, C.-z., et al., Paper based point-of-care testing disc for multiplex whole cell bacteria analysis. Biosensors and Bioelectronics, 2011. 26(11): p. 4342-4348.
18. Jokerst, J. C., et al., Development of a paper-based analytical device for colorimetric detection of select foodborne pathogens. Analytical chemistry, 2012. 84(6): p. 2900-2907.
19. Ellerbee, A. K., et al., Quantifying colorimetric assays in paper-based microfluidic devices by measuring the transmission of light through paper. Analytical chemistry, 2009. 81(20): p. 8447-8452.
20. Cheng, C. M., et al., Paper-Based ELISA. Angewandte Chemie International Edition, 2010. 49(28): p. 4771-4774.
21. Liu, X., et al. A portable microfluidic paper-based device for ELISA. in Micro Electro Mechanical Systems (MEMS), 2011 IEEE 24th International Conference on. 2011. IEEE.

While the present description has been described in connection with specific embodiments thereof, it will be understood that it is capable of further modifications and this application is intended to cover any variations, uses, or adaptations, including such departures from the present disclosure and as may be applied to the essential features hereinbefore set forth, and as follows in the scope of the appended claims.

1. A microfluidic analytical device, comprising:
a porous layer having at least one slot therein and a porous arm extending from the porous layer and pivotable about the arm's root, a distal end of the porous arm having a hydrophilic element, the porous arm being pivotable between an off position wherein the porous arm is spaced away from the slot and an on position wherein the porous arm is disposed in the slot and the hydrophilic element spans the slot to define a fluid flow path across the slot;
a heat-responsive shaped memory polymer (SMP) disposed underneath the porous layer and abutting against the porous arm, the SMP being elastically deformable in response to being heated to move the porous arm between the on and off positions; and
a heat source in heat-conducting contact with the SMP to elastically deform the SMP.

2. The microfluidic analytical device of claim 1, wherein the porous layer is selected from the group consisting of porous cellulose paper, porous hydrophilic fabrics, porous nitrocellulose paper and membrane, porous glass microfiber membrane, and porous carbon nanofiber membrane.

3. The microfluidic analytical device of claim 1, wherein the porous layer comprises fluid-impermeable barriers that define boundaries of hydrophilic regions; said hydrophilic regions comprises a fluid channel, a reagent storage zone, and a test zone; said fluid channel connects said reagent storage zone and said test zone.

4. The microfluidic analytical device of claim 3, wherein said test zone comprises an immobilized analyte binder.

5. The microfluidic analytical device of claim 3, wherein the slot disconnects said fluid channel.

6. An analytical system, comprising:
a printed circuit board having heating resistors disposed thereon, the printed circuit board being operable to energize the heating resistors to generate heat therefrom; and
a microfluidic analytical device, comprising:
a porous layer disposed over the printed circuit board, the porous layer having a slot therein and a porous arm extending from the porous layer and pivotable about the arm's root, a distal end of the porous arm having a hydrophilic element, the porous arm being pivotable between an off position wherein the porous arm is spaced away from the slot and an on position wherein the porous arm is disposed in the slot and the hydrophilic element spans the slot to define a fluid flow path across the slot; and
a heat-responsive shaped memory polymer (SMP) disposed beneath the porous layer and above the printed circuit board, the SMP abutting against the porous arm and being in heat-conducting contact with the heating resistors, the SMP being elastically deformable in response to being heated by the heating resistors to move the porous arm between the on and off positions.

7. The analytical system of claim 6, wherein the porous layer is selected from the group consisting of porous cellulose paper, porous hydrophilic fabrics, porous nitrocellulose paper and membrane, porous glass microfiber membrane, and porous carbon nanofiber membrane.

8. The analytical system of claim 6, wherein the porous layer comprises fluid-impermeable barriers that define boundaries of hydrophilic regions; said hydrophilic regions comprises a fluid channel, a reagent storage zone, and a test zone; said fluid channel connects said reagent storage zone and said test zone.

9. The analytical system of claim 8, wherein said test zone comprises an immobilized analyte binder.

10. The analytical system of claim 8, wherein the slot disconnects said fluid channel.

11. The analytical system of claim 6, further comprising a light-emitting diode (LED) and a red-green-blue color sensor for measuring the output signal of the assay.

12. The analytical system of claim 6, further comprising a liquid crystal display (LCD) screen for displaying the signal of the assay.

13. The analytical system of claim 6, further comprising a wireless communication module for transmitting assay result data to a cell phone or a computer.

14. The analytical system of claim 13, wherein the wireless communication module is a Bluetooth communication module.

15. A method of analysing a fluid analyte, comprising:
heating a porous arm to fold the porous arm into a slot, a hydrophilic portion of the porous arm spanning the slot and forming a fluid flow path across the slot; and
conveying a fluid reagent over the hydrophilic portion of the folded porous arm across the slot and into a test zone; and
analysing the fluid analyte in the test zone.

16. The method of claim 15, wherein the fluid analyte is selected from the group consisting of antigen and antibody markers.

17. The method of claim 15, for a direct or sandwich ELISA.
